(19) United States
(12) Patent Application Publication
(10) Pub. No.: US 2013/0196355 A1
(43) Pub. Date: Aug. 1, 2013

(54) Method and a kit to quantify and qualify exosomes for diagnosis of prostate cancer and prostate hyperplasia

(71) Applicant: HANSABIOMED OU, Haapsalu (EE)

(72) Inventors: Stefano FAIS, Rome (IT); Mariantonia LOGOZZI, Rome (IT); Ruggero DE MARIA MARCHIANO, Rome (IT); Desiree BONCI, Perugia (IT)

(73) Assignee: HANSABIOMED OU, Haapsalu (EE)

(21) Appl. No.: 13/803,561

(22) Filed: Mar. 14, 2013

Related U.S. Application Data
(63) Continuation-in-part of application No. 13/290,207, filed on Nov. 7, 2011.
Application No. 13/290,207 is a continuation-in-part of application No. 12/321,412, filed on Jan. 21, 2009.
(60) Provisional application No. 61/062,528, filed on Jan. 25, 2008.

(51) Int. Cl. G01N 33/68

(52) U.S. Cl. CPC G01N 33/6893
USPC 435/7.92

(57) ABSTRACT

A method and a kit are provided to quantify and qualify exosomes. Specifically the method and the kit quantify PSA-carrying exosomes for a purpose to diagnose prostate cancer and to distinguish between patients having a tumor and those having a benign prostate condition with increased blood levels of PSA. The method and the kit provide a fast, easy to use and accurate method for clinical settings.

### PRIORITY

This is a continuation-in-part application of U.S. patent application Ser. No. 12/321,412 filed on Jan. 21, 2009 claiming priority of U.S. provisional application number 61/062,528 filed on Jan. 25, 2008, and of U.S. Ser. No. 13/290,207 filed on Nov. 7, 2011, the contents of all of which are incorporated herein by reference.

### FIELD OF THE INVENTION

The present invention relates generally to the field of cancer diagnosis and prognosis. More specifically, the invention relates to a method to differentiate between prostate cancer and benign prostate conditions by means of quantifying and qualifying exosomes in human body fluids.

### BACKGROUND OF THE INVENTION

Exosomes are microvesicles of a size ranging between 30-120 nm, actively secreted through an exocytosis pathway normally used for receptor discharge and intercellular cross-talk.

Several cell types including reticulocytes, dendritic cells, B cells, T cells, mast cells, epithelial cells, and embryonic cells are known to be capable of releasing exosomes, however their increased amount in the peripheral circulation appears to be unique to pregnancy and to cancer. The primary source of circulating exosomes is the tumor. Tumor patients have been found to have very high levels of tumor derived exosomes in plasma, ascites and pleural effusions.

Molecular analyses of exosomes have demonstrated that all exosomes share certain common characteristics, including structure (delimited by lipid bilayer), size, density and general protein composition. Proteins commonly associated with all exosomes include cytoplasmic proteins such as tubulin, actin, actin-binding proteins, annexins and endolysosomal proteins such as LAMP1- and Rab-proteins, signal transduction proteins, MHC class I molecules, and heat-shock proteins (such as Hsp70 and Hsp90), and tetraspanins (such as CD9, CD81 and lysosomal proteins CD63), some of which are commonly used as exosomal markers. However, part of these proteins are not exclusively expressing on exosomes, but can be found in other sources too.

The parent patent application Ser. No. 12/321,412 claiming priority of U.S. 61/062,528, both of which are incorporated herein by reference, disclosed for the first time that Rab5 is a universal exosomal marker. Indeed Rab5 is displayed on the exosomal membrane regardless of the origin of an exosome while not found on other membrane delimited vesicles present in human biofluids.

While tumor-derived exosomes share some common exosomal proteins, they also exhibit an array of tumor related proteins, such as, but not limited to Caveolin-1, or tumor markers such as carcinoembryonic antigen or MART-1.

The elevated presence of exosomes in blood and ascites fluids of cancer patients and the over-expression of certain biomarkers has lead researchers to propose a role for exosomes in tumor marker analysis. U.S. provisional application number 61/062,528 and in the subsequent non-provisional application Ser. No. 12/321,412, both of which are incorporated herein by reference, proposed for the first time a method to quantify and qualify exosomes for use of diagnosis and prognosis of cancer.

The method suggested was based on ELISA based test using anti-Rab5 as primary antibody, and example wise anti-CD63 or anti-caveolin 1 antibodies as secondary antibodies. Later U.S. Pat. No. 7,897,356 disclosed a method of characterizing prostate cancer in a subject by identifying a biosignature on an exosome by determining presence or level of CD9, CD63, or CD81 protein from exosomes, determining presence or level of PSMA and/or PCSA protein from exosomes, determining the presence or level of B7H3 and/or EpCam protein from the exosomes and then comparing the levels with a reference.

Prostate cancer is the second leading cause of death from cancer among men. The confined tumors can be successfully treated by surgery only before tumor spreading, but no effective treatment has yet been identified to cure the metastatic forms of prostate cancer. Moreover, the complexity in recruiting tumor tissue from castration-resistant or metastatic patients represents a limitation for follow up monitoring and therapy decision based on molecular aspect.

The currently used methods to diagnose organ-confined disease typically include measuring plasmatic levels of prostate specific antigen (PSA) in single patients and/or digital rectal examination followed by local biopsies. The PSA is a glycoprotein with a molecular weight of 33-34 kD. After leakage into the blood, the enzymatically active form of PSA combines with serum antiproteases like a-1 antichymotrypsin (a-1 ACT) and a-2 macroglobulins (a-2 MG), whereas the enzymatically inactive form remains free in the blood and both can be detected by available PSA immunoassays.

PSA test is currently commonly in use to monitor prostate cancer in men. The test measures the level of PSA in a man's blood. The blood level of PSA is often elevated in men with prostate cancer. However, the flaw with this test is that in addition to prostate cancer, a number of benign conditions can cause a man's PSA level to rise. The most frequent benign prostate conditions that cause an elevation in PSA level are prostatitis (inflammation of the prostate) and benign prostatic hyperplasia (enlargement of the prostate). Elevated levels are also seen in patients after manipulating prostate gland by digital rectal examination, transrectal ultrasonography, catheterization, prostatic biopsy and after radical prostatectomy. Therefore, the PSA test may give false-positive results.

In order to optimize the use of PSA for detection of prostate cancer several new concepts have developed. These include PSA density, PSA velocity, age specific reference ranges and percentage of free PSA. Among the various modalities, the percentage of free PSA has shown potentially encouraging results. It has been proposed that the proportion of serum free PSA is significantly higher in patients with nodular hyperplasia than in patients with prostate cancer. In fact, the cancer cells, due to structural changes in PSA molecule, may produce lower proportion of free PSA. In case of non-neoplastic prostate disease the ratio of PSA free and total PSA is close to 1, while it is lower for patients with tumors. Even if the percentage of free PSA may be useful in discriminating between patients with non-neoplastic prostate disease and prostate cancer, the PSA screening still misses to discriminate benign prostatic hypertrophy and indolent forms from aggressive tumors.

The PSA test is also used to monitor patients who have a history of prostate cancer to see if their cancer has recurred (come back). If a man's PSA level begins to rise after prostate cancer treatment, it may be the first sign of a recurrence. Such a “biochemical relapse” typically appears months or years before other clinical signs and symptoms of prostate cancer recurrence. However, an elevated PSA measurement in a patient who has a history of prostate cancer does not always mean that the cancer has come back. The PSA test may thus give false positive results.

Prostatectomy is partial or total removal of prostate gland. Partial or radical prostatectomy is generally effective in treating prostate cancer that has not spread. After a prostatectomy the blood PSA levels will drop almost to zero if the surgery successfully remove the cancer and the cancer has not spread. However, if cancer has spread, advanced cancer may develop even after the prostate has been removed and the PSA levels are low. Thus PSA test may give a false negative result.

Even if PSA level evaluation has improved the identification of early-stage tumors, it has on the other hand caused over-diagnosis, over-treatment and rising cost of public national health. Thus, new tools for cancer detection, capable of providing an accurate individual risk assessment and markers to drive therapy decision are needed. There is a need for reliable and easy method for diagnosing prostate cancer and for discrimination between cancerous and benign stages.

### SUMMARY OF THE INVENTION

This invention provides a solution for the above described flaws of the currently used technology and others.

It is an object of this invention to provide a fast, easy to use, and accurate method for diagnosing prostate cancer.

It is a further object of this invention to provide a fast, easy to use, and accurate method for discriminating between prostate cancer and benign conditions of prostate.

It is an object of this invention to provide a method to quantify and qualify exosomes in human cell derived samples or in body fluids, said method comprising the steps of: capturing total exosomes of the human cell derived sample or body fluid with a primary antibody against a protein exclusively present on the outer surface of exosomes; detecting prostate tumor related exosomes from the captured total exosomes with a detection antibody, said detection antibody being anti-PSA; allowing an enzyme linked secondary antibody to react with the detection antibody; adding a substrate; and detecting the reaction and quantifying prostate tumor related exosomes.

It is another object of this invention to provide a method to distinguish between prostate hyperplasia and prostate cancer of a patient having an enlarged or otherwise abnormal prostate gland, said method comprising the steps of: collecting a body fluid or cell derived sample from the patient and from a healthy person with a normal prostate gland for a control sample; capturing total exosomes of the samples with a primary antibody against a protein exclusively present on the outer surface of the exosomes; detecting exosomes carrying PSA protein on their outer membrane surface with an anti-PSA detection antibody; allowing an enzyme linked secondary antibody to react with the detection antibody; adding substrate; detecting the reaction and quantifying the tumor related exosomes based on the reaction; comparing the quantity of tumor related exosomes in the patient's sample with the quantity in the control sample; wherein a diagnosis of prostate hyperplasia is given when the quantity PSA expressing exosomes is similar in both of the samples and a diagnosis of cancer is given when the quantity of PSA expressing exosomes in patient's sample is increased as compared to the control sample.

It is yet another object of this invention to provide a test kit for diagnosis and prognosis of a prostate tumor, said kit comprising: a primary antibody preparation for capturing total exosomes from a body fluid or cell derived sample of a patient, wherein the primary antibody is against a protein exclusively present on outer surface of exosomes; a detection antibody preparation for detecting prostate tumor related bound exosomes, wherein the detection antibody is anti-PSA; an enzyme linked secondary antibody preparation for reaction with the detection antibody; a substrate for the enzyme; and a control sample.

### BRIEF DESCRIPTION OF THE DRAWINGS

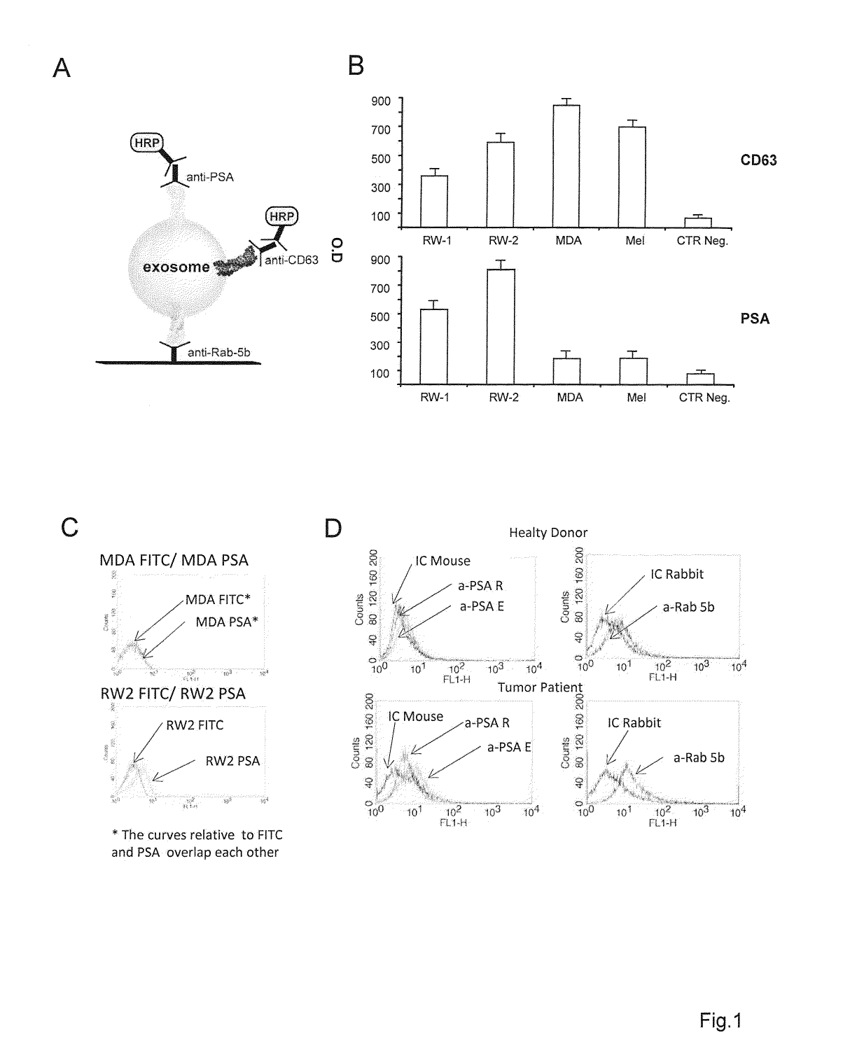

FIG. 1 Exotest and cytofluorimetric analysis confirmed PSA expression on tumor-derived exosomes.
A) An illustration showing the principle of the ELISA based test according to this invention (Exotest®). The wells of the test kit are coated with Anti-Rab-5-antibody which is the primary antibody capturing total exosomes. Anti-PSA or CD63 antibodies are included as secondary antibodies and labeled with the enzyme horseradish peroxidase (HRP).
B) Exosomes isolated from RWPE-1(non-neoplastic prostate cells), RWPE-2 (early prostate tumor), MDA231 (Breast cancer) an Mel-1 (melanoma cancer) were analyzed for PSA expression by Exotest of FIG. 1A. The values were reported as arbitrary values and reaction buffer was used as negative control (Ctr.neg.).
C) Cytofluorimetric analysis of PSA expression on exosomes isolated from RWPE-2 and MDA231 cells and coated on aldehyde/sulfate latex beads for 12 hours. One representative experiment of three was reported. A control antibody was used as negative staining control. D) Cytofluorimetric analysis of PSA expression on aldehyde/sulfate latex beads-coated exosomes isolated from three patients with tumors and healthy donors. One representative experiment is shown of each. Anti-Rab-5b antibody was used as exosomes control. A control antibody was used as negative staining control.

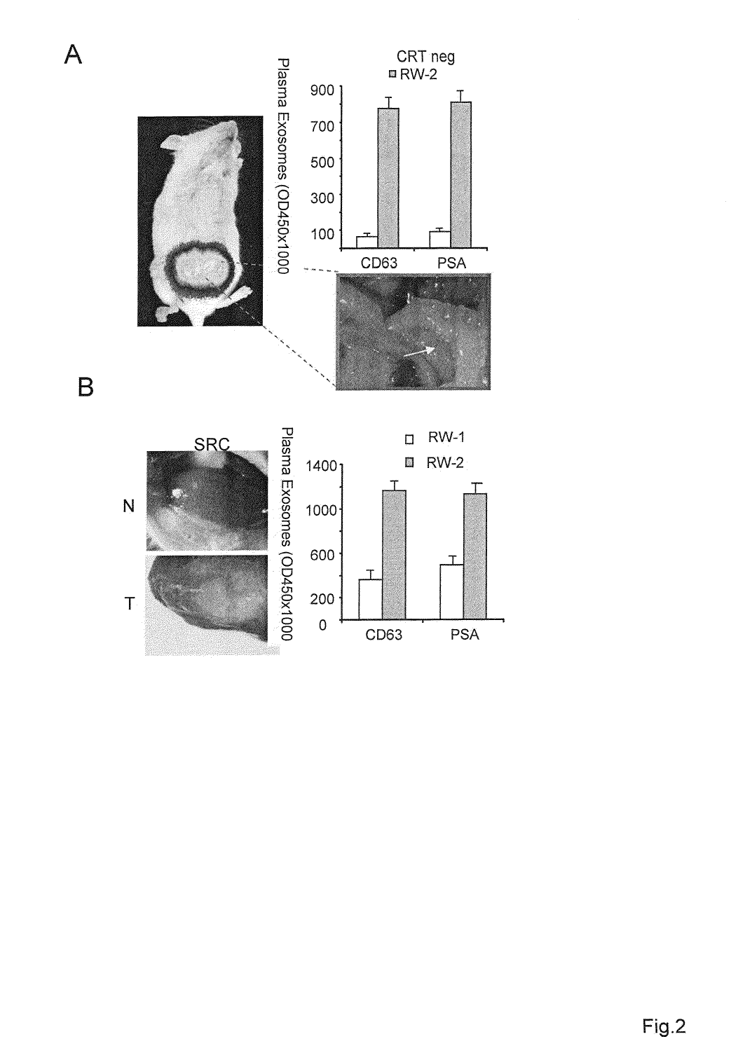

FIG. 2 PSA-positive exosomes robustly distinguished cancers and hypertrophies.
A) RWPE-2, infected with luciferase gene, were inoculated in anterior prostate of NSG mice and evaluated three weeks after injection by IVIS system. One representative image is shown. Evaluation of exosomes positive for CD63 and PSA in plasma of inoculated mice and analyzed by Exotest of FIG. 1A. PBS injected mice were used as negative test control. Three independent experiments were conducted and the data is shown as mean ±S.D.
B) Non-neoplastic RWPE-1(RW-1) and tumor RWPE-2 (RW-2), transduced with EGFP reporter gene, were inoculated under renal capsule (SRC) and analyzed by stereomicroscopy system. Evaluation of exosomes positive for CD63 and PSA in plasma of inoculated mice and analyzed by Exotest of FIG. 1A. Three independent experiments were conducted and the data was were reported as mean ±S.D.

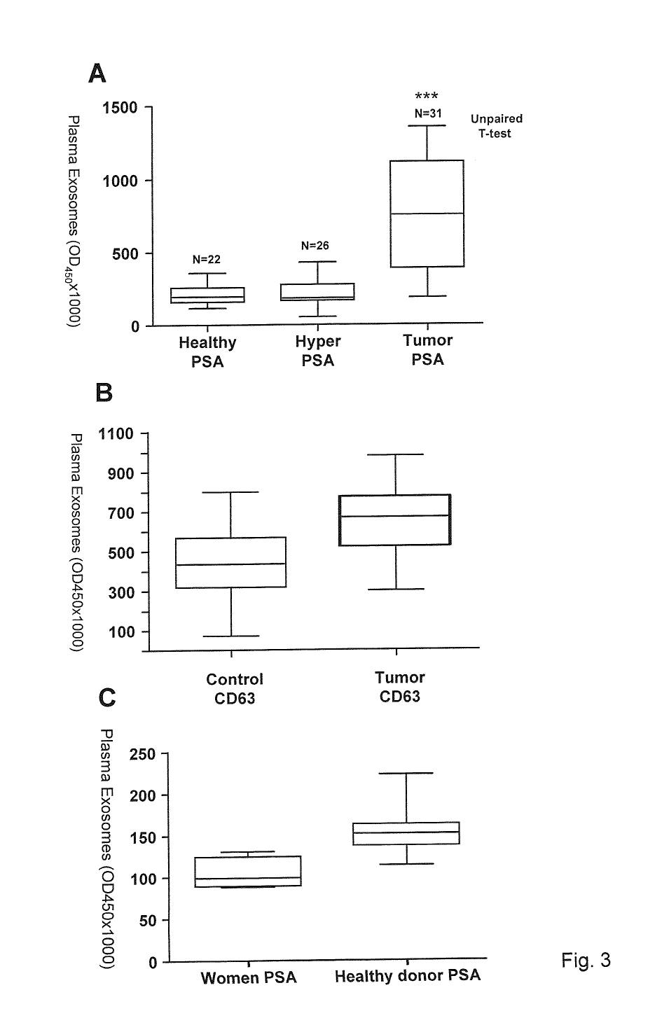

FIG. 3 A) Exosomes isolated from patients' plasma and evaluated for PSA expression by the method of this invention. 22, 26 and 31 patients were analyzed for healthy, hypertrophy and cancer groups respectively. Optical density values are shown. *** represents P<0.001 as evaluated by unpaired t-test.
B) Exosomes isolated from patients'plasma and evaluated for CD63 expression by the method of this invention. 22, 26 and 31 patients were analyzed for healthy, hypertrophy (Control) and cancer groups respectively. Optical density values are shown as arbitrary units.
C) PSA positive exosomes (PSA-EX) evaluation in plasma of 12 women and 22 healthy donors performed by the method of this invention. Optical density values are shown as arbitrary units.

### DETAILED DESCRIPTION OF THE INVENTION

### Definitions

ExoTest® is a trademarked ELISA-based test that was first described and claimed in the U.S. provisional patent application number 61/062,528 and subsequent U.S. Ser. No. 2009/0220944, both of which are incorporated herein by reference. ExoTest platform comprises ELISA plates pre-coated with antibodies against housekeeping exosome proteins enabling specific capture of exosomes from different biological samples, including cell culture supernatants and human biological fluids. Quantification and characterization of exosomal proteins is subsequently performed by using appropriate detection antibodies against exosome associated antigens that can be either common for all exosomes or cell type- or cell condition specific.

Exosomes are microvesicles of a size ranging between 30-120 nm, actively secreted in the extracellular environment by normal as well as tumor cells.

The invention is now described by means of non limiting examples.

### EXAMPLE 1

### Isolation of Exosomes and Labeling of the Exosomes

Exosomes Isolation

Exosomes were purified by three successive centrifugations at 300×g (5 min), 1200×g (20 min) and 10.000×g (30 min) to eliminate cells and debris, followed by centrifugation for 1 h at 100 000×g. The exosome pellets were washed once in a large volume of PBS, centrifuged at 100.000×g for 1 h and re-suspended in 50-200 μl of PBS. To obtain plasma exosomes, the classical protocol was modified because of plasma viscosity and protein and lipid abundance compared with the cell supernatant. After the separation from total blood, plasma was centrifuged for 30 min at 500×g, 45 min at 12 000×g and 2 h at 110 000×g. Pellets were re-suspended in a large volume of PBS, filtered on a 0.22-μm filter (Millipore) and centrifuged at 110 000×g for 1 h. Microvesicle pellets were washed once in a large volume of PBS, centrifuged at 110 000×g for 1 h and re-suspended in 50-200 μl of PBS. The amount of 110.000×g pellet proteins recovered are measured by Bradford assay (Bio-Rad). Exosomes were used as fresh preparation for immunoisolation by sytofluorimetry analysis or ELISA-based test, the principal of which is shown in FIG. 1A.

### Exosomes Labeling

For immunoisolation and FACS analysis of microvesicles exosome pellets were resuspended in 10 μl of 4-μm-diameter aldehyde/sulfate latex beads (Interfacial Dynamics, Portland, Oreg., USA) were incubated with purified anti-CD63 or prostate specific protein hAbs at room temperature in a small volume (50 μl). After 15 min, the volume was made up to 400 μl with PBS and incubated overnight at 4° C. under gentle agitation. Exosome-coated beads were washed twice in

FACS washing buffer (1% BSA and 0.1% NaN3 in PBS) and re-suspended in 400 μl FACS washing buffer, stained with fluorescent antibodies and analyzed on a FACSCalibur flow cytometer (BD Biosciences) and CellQuest software.

### EXAMPLE 2

### Prostate Specific Antigen PSA is Expressed on Exosomes Detected by Exotest

The presence of PSA on exosomes was further validated by using an ELISA test (Exotest®) where the primary capturing antibody was RAB5b antibody capturing total exosomes of the sample and the secondary antibody was either PSA-antibody or CD-63 antibody. Exosomes isolated from a normal cell line, RWPE-1 and RWPE-2 cells were loaded on RAB5b-antibody coated plates (FIG. 1A). We used a breast and melanoma cancer cells as PSA negative controls. RAB5b attached exosomes were incubated with antibodies against either CD63 or PSA and evaluated by peroxidises enzymatic reaction (FIG. 1A). Tumor cell lines released significantly higher amount of exosomes (CD63+) as compared to non-neoplastic cells (FIG. 1B), while PSA-bearing exosomes were exclusively detected in the supernatant of non-neoplastic prostate and tumor cell lines (FIG. 1B).

This data was highly consistent with that obtained by FACS analysis of aldehyde/sulfate latex beads, following overnight exosome coating and staining with an additional anti-PSA antibody (FIG. 1C, D). Results confirmed PSA expression on exosomes surface of both RWPE-2 and patients' plasma (FIG. 1C, D). Breast cancer cells and healthy man donors were used as negative controls. Rab5b staining on plasma samples was used as control.

### EXAMPLE 3

### Detection of PSA Exosomes in Mice Inoculated With RWPE-2 Cells

In vivo mice models

Six-eight week old male NOD.Cg-Prkdcscid I12rgtm1 Wj1/SzJ, NSG mice (Charles River Laboratoty, Wilmington, Mass.) were used for experiments.

Sub-renal capsule injection: To perform renal capsule injection, a skin incision of approximately 1 cm was made along the dorsal midline of an anesthetized mouse. Then, an incision was made in the body wall slightly shorter than the long axis of the kidney. After exteriorization of the kidney, the capsule was lifted from renal parenchyma to allow an injection or a 2-4 mm incision to injected 105 cells with or without matrigel. Three weeks from injection, mice were analyzed and blood samples withdrawals were performed.

Orthotopic injection: For orthotopic injection, abdominal wall muscles were incised, and the bladder and seminal vesicles were delivered through the incision to expose the prostate. 105 cells were injected via a 0.3 mm needle directly into the anterior prostate. The incision was closed using a running suture of 4-0 silk. Cells were infected with luciferase gene and monitored with IVIS Imaging System 100 Series (Xenogen). All animal procedures were performed according to protocols approved by the ISS Animal Care Committee.

A set of experiments were performed by inoculating RWPE-2 cells into the anterior prostate lobe of NSG mice and PSA positive exosome were quantified 9 weeks after injection into the plasma by capturing total exosomes with antiRab5b antibody and selectively capturing PSA positive exosomes with antiPSA-antibody on an Exotest kit. CD63 positive exosomes were captured from the total exosomes by using CD63 antibody as secondary antibody. PBS inoculated mice were used as internal control. Data showed the presence of human CD63 and PSA-positive exosomes in the mice injected with tumor cells (FIG. 2A).

Since it is reported that tumors release marked levels of exosomes, we wanted to test the sensitivity of our approach comparing the tumor RWPE-2 and non-neoplastic RWPE-1 cells. We injected both cell lines into an highly permissive and vascularised site, under the renal capsule (SRC) (FIG. 2B) and we evaluated PSA positive plasmatic exosomes 4 weeks after injection by Exotest. Results showed that tumor cells released exosomes 2 fold more than non-neoplastic cells and that this system robustly distinguished aggressive and non-neoplastic cells (FIG. 2B).

This data supports that the method of this invention guarantees high sensitivity to distinguish between tumor and non-neoplastic cells.

### EXAMPLE 4

### PSA Positive Exosomes Identify Patients With Prostate Cancer

In order to investigate the diagnostic significance of our data, we collected plasmatic samples from a large cohort of patients, representative of all phases of prostatic neoplastic disease. In detail, plasma was collected from 31 patients with diagnosed cancers (Tumor) and 26 patients with non-neoplastic prostatic disease (Hypertrophy). A group of healthy men (22) (Healthy) were used as control population. From the plasma samples exosomes were isolated by ultracentrifugation and were analyzed for the presence of either CD63 or PSA. Plasma collected from patients with tumor showed higher levels of exosomes as compared to healthy donors as analyzed by the CD63 Exotest analysis (FIG. 3B). However, patients with cancer showed a significant increase in the level of PSA-positive exosomes than hypertrophic patients (FIG. 3A). Exosomes isolated from six women were used as exosomal PSA negative controls (FIG. 3C). Setting a cut-off at 500 as arbitrary unit value, chosen as maximum value in the hypertrophic patients value distribution (in FIG. 3A), PSA-positive exosomes level well discriminates the patients with tumors from ones with hypertrophy as determined by Exotest analysis of this invention. This data offers a new and alternative diagnostic tool by passing the conventional screening test limits with a non-invasive approach for patients.

### EXAMPLE 5

### Quantification of Exosomes by Using Unfractionated Biological Fluids

In order to provide a fast and simple test for clinical purposes we verified that the test can be used for exosome detection in unfractionated biological fluids that would allow an easy and reproducible analysis avoiding the steps of ultracentrifugation. We compared the detection and quantification of PSA exosomes from unfractionated samples (human plasma, urine samples) and purified exosomes samples. The presence of PSA-exosomes in unfractionated samples was detectable by ExoTest (results not shown). The results suggest that ExoTest is useful and reliable in clinical setting using whole plasma or urine samples and avoiding the complex and time consuming procedures of exosome purification. Thus the test disclosed here provides an easy to use, fast and patient friendly test for accurately diagnose prostate cancer and to discriminate it from benign conditions where plasma PSA levels may be increased.

What is claimed is:

1. A method to quantify and qualify exosomes in human cell derived samples or in body fluids, said method comprising the steps of:
a) capturing total exosomes of the human cell derived sample or body fluid with a primary antibody against a protein exclusively present on outer surface of exosomes;
b) detecting prostate tumor related exosomes from the captured total exosomes with a detection antibody, said detection antibody being anti-PSA;
c) allowing an enzyme linked secondary antibody to react with the detection antibody;
d) adding a substrate; and
e) detecting the reaction and quantifying prostate tumor related exosomes.

2. The method of claim 1, wherein the primary antibody is anti -Rab 5 antibody.

3. The method of claim 1, wherein the primary antibody is anti-Rab 5b antibody.

4. The method of claim 1, wherein the method includes a step of purifying an exosome preparation from the human cell derived sample or body fluid, and step a) is performed with purified exosomes.

5. The method of claim 1, wherein body fluid is human plasma sample or urine.

6. The method of claim 1, wherein the body fluid is received from a healthy person as a control sample and from a patient suspected to have a prostate tumor, and the detected quantity of prostate cancer related exosomes in patient's sample is compared with the quantity of prostate cancer related exosomes in the control, and wherein an increase in the quantity is used as an indicator of prostate tumor.

7. A method to distinguish between prostate hyperplasia and prostate cancer of a patient having an enlarged or otherwise abnormal prostate gland, said method comprising the steps of:
a) collecting a body fluid or cell derived sample from the patient and from a healthy person with a normal prostate gland for a control sample;
b) capturing total exosomes of the samples with a primary antibody against a protein exclusively present on outer surface of exosomes;
c) detecting exosomes carrying PSA protein on their outer membrane surface with an anti-PSA detection antibody;
d) allowing an enzyme linked secondary antibody to react with the detection antibody;
e) adding a substrate;
f) detecting the reaction and quantifying the tumor related exosomes based on the reaction;
g) comparing the quantity of tumor related exosomes in the patient's sample with the quantity in the control sample, wherein a diagnosis of prostate hyperplasia is given when the quantity PSA expressing exosomes is similar in both of the samples and a diagnosis of cancer is given when the quantity of PSA expressing exosomes in patient's sample is increased as compared to the control sample.

8. The method of claim 7, wherein the primary antibody is anti-Rab 5 antibody.

9. A method to diagnose prostate cancer, said method comprising the steps of:
a) collecting a body fluid or cell derived sample from a patient and from a healthy person as a control sample;
b) capturing total exosomes of samples with a primary antibody against a protein exclusively present on outer surface of exosomes;
c) detecting prostate tumor related exosomes from the captured total exosomes with an anti-PSA detection antibody;
d) allowing an enzyme linked secondary antibody to react with the detection antibody;
e) adding a substrate;
f) detecting the reaction and quantifying the tumor related exosomes based on the reaction; and
g) comparing the quantity of tumor related exosomes in the patient's sample with the quantity in the control sample and diagnosing cancer when the quantity of in patient's sample is increased as compared to the control sample.

10. The method of claim 9, wherein the primary antibody is anti-Rab 5 antibody.

11. A non-invasive method to monitor prostate tumor growth, said method comprising the steps of:
a) periodically taking a body fluid sample of a patient;
b) capturing total exosomes of the exosome preparations with a primary antibody against a protein exclusively present on outer surface of exosomes,
c) detecting the bound exosomes with an anti-PSA detection antibody,
d) allowing an enzyme linked secondary antibody to react with the detection antibody;
e) adding a substrate;
f) detecting the reaction and quantifying the tumor related exosomes based on the reaction; and
g) drawing a correlation between quantity of detected exosomes in step f) and progress of growth of prostate tumor.

12. The method of claim 11, wherein the primary antibody is anti-Rab 5 antibody.

13. A test kit for quantifying and qualifying exosomes in human cell derived samples or in body fluids, said kit comprising:
a) a primary antibody preparation for capturing total exosomes from a body fluid or cell derived sample of a patient, wherein the primary antibody is against a protein exclusively present on outer surface of exosomes;
b) a detection antibody preparation for detecting prostate tumor related bound exosomes, wherein the detection antibody is anti-PSA;
c) an enzyme linked secondary antibody preparation for reaction with the detection antibody;
d) a substrate for the enzyme; and
e) a control sample.

14. The kit of claim 13, wherein the primary antibody is antiRab5.

15. The kit of claim 13, wherein the kit is for diagnosis and prognosis of prostate tumor.
